(19) United States
(12) Patent Application Publication
(10) Pub. No.: US 2011/0262564 A1
(43) Pub. Date: Oct. 27, 2011

(54) Treatment of Cancer with Selenium Nanoparticles

(76) Inventors: Xueyun Gao, Beijing (CN); Ling Kong, Beijing (CN)

(21) Appl. No.: 12/818,604

(22) Filed: Jun. 18, 2010

Related U.S. Application Data
(60) Provisional application No. 61/327,056, filed on Apr. 22, 2010.

(51) Int. Cl. A61K 33/04; C12N 5/00; A61P 35/00

(52) U.S. Cl. 424/702; 435/375

(57) ABSTRACT

Novel chemopreventive and chemotherapeutic cancer treatment method using elemental selenium nanoparticles. Cancer cells, especially androgen dependent prostate cancers are exposed to selenium from elemental selenium nanoparticle treatment, and apoptosis is induced in the cancer cells.

### CROSS-REFERENCE

Priority is claimed from U.S. Provisional Application 61/327,056 filed on Apr. 22, 2010, the entirety of which is hereby incorporated by reference.

### DESCRIPTION OF RELATED ART

The present application relates to cancer treatment, and more particularly to treatment of cancer using selenium nanoparticles prepared with crude peptone solution.

Note that the points discussed below may reflect the hindsight gained from the disclosed inventions, and are not necessarily admitted to be prior art.

Selenium is an essential trace mineral for life. It is also a toxic metal when consumed in excess. Selenium is required to activate various key enzymes, including the antioxidant glutathione peroxidase, the metabolic enzyme thioredoxin reductase, and the thyroid-hormone-activating enzyme iodothyronine deiodinase.

It is generally accepted that the antioxidant activity of selenium is linked with its anticancer effect. A connection between cancer incidence and low levels of selenium in the blood was recognized by epidemological studies as early as 1969. It was also noted that breast cancer rates were low in areas where selenium levels in the soil and food were high and high in areas where selenium levels were low. The same correlation was found between death rates and selenium levels, and similar correlations were found in animal studies. Selenium compounds have therefore been used as chemopreventive and chemotherapeutic agents.

In humans, it has been observed that a 200 microgram daily supplement given for four years led to a significant reduction in cancer deaths and reduction in the incidence of prostate, lung, colorectal and some types of skin cancer in comparison to the control group who received 85 microgram per day in their diet. Since the known selenoenzymes are saturated with 90 microgram daily of selenium, it is proposed that an additional anticancer mechanism is in play in addition to the antioxidant activity of selenium.

Different forms of selenium have also been shown to be of different level of effect. Inorganic forms of selenium such as sodium selenite have proven more effective at fighting cancer than the commonly used organic form, selenomethionine, yet selenomethionine was more effective at increasing selenium tissue levels and glutathione peroxidase activity. Because cells cannot distinguish selenomethionine from the essential amino acid methionine, some selenomethionine becomes incorporated into general body proteins, increasing tissue selenium levels.

For either selenomethionine and sodium selenite, there are shortcomings. Selenomethionine general proteins have no anticancer activity and sodium selenite is more frequently metabolized to the toxic metabolite hydrogen selenide (H₂Se). Hydrogen selenide does have anticancer effects but it is more toxic. Its primary mode of killing cancer cells (and at high levels, normal cells) is through the process of cell necrosis. Cell necrosis provokes inflammation and may kill healthy cells along with cancer cells.

Further, in generally toxicity, the inorganic forms of selenium are more toxic than the organic form. Researchers have noted that organic forms of selenium are toxic at levels in the vicinity of 3,500 micrograms (3.5 milligrams) daily while inorganic forms of selenium may be toxic at 900 microgram per day.

As nutritionally-oriented physicians may use as much as 900 to 2,000 microgram selenium daily as part of a comprehensive cancer treatment protocol, more recent anticancer treatment therefore has focused on other compounds, such as methyselenocysteine (SeMC). Methyselenocysteine is an organoselenium compound found naturally in some vegetables. SeMC is also more bio-absorbable than other forms. Unlike selenome-thionine, which is incorporated into proteins in place of methionine, SeMC is not incorporated into any proteins, thereby being fully available for the synthesis of selenium containing enzymes, such as glutathione peroxidase. However, purification of SeMC is a complicated process.

Recent evidence has shown that elemental selenium nano-particles exhibit novel properties both physically and biologically. Although elemental selenium metal powder in the redox state of zero is not soluble and biologically inert, elemental selenium nano-particles can be well ingested and uptaken by cells and are biologically active.

Recently neonatal elemental selenium atoms in the forms of nano-particles of 20-60 nm size prepared with pure bovine albumin protein have been shown to exhibit comparable efficacy in up-regulating selenoenzymes as does inorganic selenium selenite, but much less toxic than selenite, selenomethionine or Se-methylselenocysteine. See Zhang, J. et al., “Comparison of short-term toxicity between Nano-Se and selenite in mice,” 2005, Life Sci., 76, 1099-1109; Zhang, J., et al., “Elemental selenium at nano size as a potential chemopreventive agent with reduced risk of selenium toxicity: comparison with Se-methylselenocysteine in mice,” 2008, Toxicological Sciences, 101, pp 22-31.

Nano-selenium particles prepared with pure bovine albumin protein are also shown to reduce Lewis lung cancers in mouse. See Gao, X., et al., “The effect of nano red elemental selenium on the Lewis Lung Cancer planted in C₅₇ Mice,” 2000, China Public Health, vol 16, No. 2, pp 109-200. Moreover, selenium nano-particles are shown to enhance the immune system while selenite has no effect. See Gao, X., et al., “The effect of nano red elemental selenium on immune function of mice,” 2000, China Public Health, vol 16, No. 5, pp 421-422.

Nano-selenium particles therefore as a much less toxic, more potent form of selenium have great potential in comprehensive cancer treatment and cancer chemoprevention. However, nano-selenium particles are currently prepared with pure bovine albumin protein which makes the nano-selenium particles expensive. The cost factor is therefore prohibitive for nano-Se to be used as a replacement of the inorganic or organic forms of selenium in cancer treatment. Novel preparation of nano-Selenium with comparable effects is needed.

### SUMMARY

The present application discloses that the selenium nanoparticles prepared with crude peptones are more effective than the most potent organic selenium form, methylseleninic acid (MSA) in killing cancer cells and directly induction of apoptosis.

In one embodiment, cancer cells are exposed to selenium atoms of selenium nanoparticles prepared with crude peptone solution.

In another aspect of an embodiment, in a comprehensive chemotherapeutic program, a patient's prostate cancer cells are exposed to selenium atoms of selenium nanoparticles prepared by using crude peptone solution.

In another aspect of an embodiment, in a chemopreventive program, patients are administered pharmaceutical compositions containing selenium nanoparticles prepared by using crude peptones solutions.

For the first time it is shown that selenium nanoparticles can directly induce apoptosis in cancer cells, especially androgen dependent prostate cancer cells, more effective than MSA, the most potent organic selenium form. As they are less toxic, and also cost effective, selenium nanoparticles prepared with crude peptone solutions makes them a good candidate for cancer chemotherapy and chemopreventive programs.

### BRIEF DESCRIPTION OF THE DRAWINGS

The disclosed inventions will be described with reference to the accompanying drawings, which show important sample embodiments of the invention and which are incorporated in the specification hereof by reference, wherein:

FIGS. 1A and 1B show a graphical representation of the effect of nano-Se on human prostate carcinoma LNCaP and DU145 cell proliferation.

FIG. 2 shows the effect of nano-Se on the induction of apoptosis of LNCaP cells.

FIGS. 3A and 3B show a comparison between the effect of Nano-Se on and the effect of MSA on LNCaP cell proliferation.

FIGS. 4A and 4B show a comparison of the effects of Nano-Se and MSA on cell growth in androgen-dependent human prostate cancer cell line DU145.

FIGS. 5A and 5B show a comparison of the effects of Nano-Se and MSA on cell growth in androgen-independent human prostate cancer cell line MCF-7.

FIGS. 6A and 6B show a comparison of the effects of Nano-Se and MSA on cell growth in the human breast cancer cell line A549.

FIGS. 7A and 7B show the effect of Nano-Se on androgen regulated transcription activity in HEK 293.

FIG. 8 shows the androgen dependence of the effect of nano-se nanoparticles on prostate specific antigen (PSA) mRNA levels in LNCaP cells.

FIG. 9 shows the effect of Nano-Se on androgen receptor (AR) mRNA levels and protein levels in LNCaP cells.

### DETAILED DESCRIPTION OF SAMPLE EMBODIMENTS

The numerous innovative teachings of the present application will be described with particular reference to presently preferred embodiments (by way of example, and not of limitation). The present application describes several inventions, and none of the statements below should be taken as limiting the claims generally.

For simplicity and clarity of illustration, the drawing figures illustrate the general manner of construction, and description and details of well-known features and techniques may be omitted to avoid unnecessarily obscuring the invention. Additionally, elements in the drawing figures are not necessarily drawn to scale, some areas or elements may be expanded to help improve understanding of embodiments of the invention.

The terms “first,” “second,” “third,” “fourth,” and the like in the description and the claims, if any, may be used for distinguishing between similar elements and not necessarily for describing a particular sequential or chronological order. It is to be understood that the terms so used are interchangeable. Furthermore, the terms “comprise,” “include,” “have,” and any variations thereof, are intended to cover non-exclusive inclusions, such that a process, method, article, apparatus, or composition that comprises a list of elements is not necessarily limited to those elements, but may include other elements not expressly listed or inherent to such process, method, article, apparatus, or composition.

The chance of a man developing invasive prostate cancer during his lifetime is 1 in 6 or 15.4%. At the age of 50, a man has a 42% chance of developing prostate cancer and 2.9% of dying from the disease. Prostate cancer development in human proceeds through multiple steps: prostatic intraepithelial neoplasia, prostate cancer in situ, invasive, and hormone-dependent or -independent metastatic cancer. Up to 80% of patients demonstrate a temporary hormonal responsiveness lasting a median of 12-18 months, before continued tumor growth is evident despite castrate levels of testosterone. Once androgen-independent growth is established, median life expectancy is 9-12 months.

Hormonal dependence of prostate cancer was first recognized by Huggins and Hodges in 1941. They showed that the removal of androgens leads to regression of prostate cancer. This recognition was a basis for therapy for non-organ-confined prostate tumors. Despite the initial efficacy of androgen deprivation therapy, tumor cells eventually relapse into hormone-refractory prostate cancer. See Mimeault M and Batra S K, “Recent advances on multiple tumorigenic cascades involved in prostatic cancer progression and targeting therapies,” 2006, Carcinogenesis, vol 27, pp 1-22.

Recent reports have shown that the androgen receptor (AR) is the key determinant of the molecular changes required for driving prostate cancer cells from an androgen-dependent to an androgen-independent state. AR amplification with concomitant overexpression of AR that increases the sensitivity of prostate cancer cells to low levels of androgens resulted in the development of androgen-independent prostate cancer. See Linja, et al., “Amplification and over-expression of androgen receptor gene in hormone-refractory prostate cancer,” 2001, Cancer Res., vol 61, pp 3550-3555. The fact that knocking down AR expression by shRNA or siRNA in AR-positive cell lines resulted in apoptotic death indicates that down-regulating of AR expression may be used for therapeutical purposes. See Cheng et al., “Short hairpin RNA knockdown of the androgen receptor attenuates ligand-independent activation and delays tumor progression,” 2006, Cancer Res., vol 66, pp 10613-10620.

Evidence shown in this application indicates that selenium nanoparticles, prepared with crude peptones, can inhibit AR transcription and protein expression, and induce apoptosis in prostate cancer cells. Selenium nano-particles are therefore a good candidate to be used for prostate cancer treatment and prevention.

### EXAMPLES

### Preparation of Selenium Nano-Particles

Sodium selenite (99.99%), L-cysteine (99.99%), peptones from soybean (80%) were purchased from Sigma, and stored in a dry box. Water was distilled prior to use. Fifty grams of peptone from soybean were added to 1000 ml of 100 mM sodium selenite solution. Then L-cysteine powder was continuously added to the solution with stirring until a final concentration of 400 mM. The resulted mixture was stirred at 25° C. for 10 hours. Then sodium ions and oxidized-L-cysteine was removed by dialysis against water, and a solution of amorphous selenium nanoparticles is obtained.

### Example 1

To determine the cell viability effect of nano-Se in human prostate cancer cells, androgen-dependent LNCaP cells and androgen-independent DU145 cells were treated with 0, 1, 5, 10, 50 and 100 μM of Nano-Se particle suspensions for 24 and 48 hrs respectively.

LNCaP (Lot#1735217), and DU-145 (Lot#1145858) human prostate cancer cell lines were purchased from the American Type Culture Collection (ATCC, Manassas, Va., USA). Cells were maintained in 1640 culture medium (Invitrogen Life Technologies, USA) supplemented with 10% (v/v) fetal bovine serum and antibiotics.

For cell viability assay, cells were seeded into 96-well plates at a density of 1×10⁴ cells/well, and 40-50% confluent cells were subsequently treated with nano-Se nanoparticles (1 to 100 μM) or distilled water for 24 or 48 hrs respectively. Treated cells were then collected and measured by Cell Counting Kit-8 (CCK-8) system (Dojindo Laboratory, Kumamoto, Japan). Briefly, CCK-8 solution (10 μl per 100 ul of medium in each well) was added, the plates were then incubated at 37° C. for 1 hour, and the absorbance of each well was read at 450 nm using a microplate reader. Values were shown as means±SE of quadruplicated determinations of a representative experiment (repeated three times). Differences between groups were analyzed using Student's test with significance assumed at p<0.05. All statistical analyses were performed using Origin75 software.

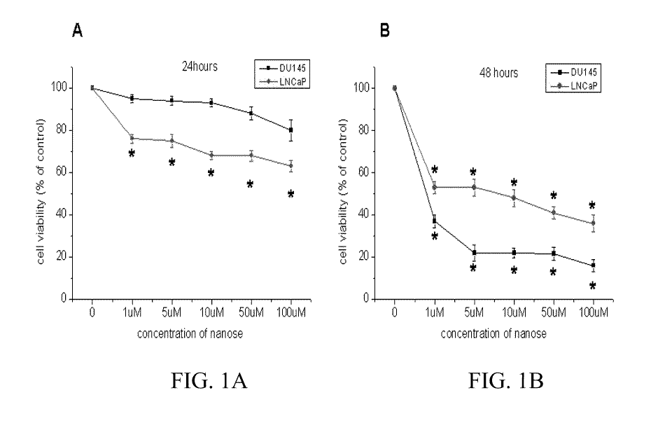

As shown in FIGS. 1A and 1B, nano-Se nanoparticles decreased the viability of both LNCaP and DU145 cells in concentration dependent manner, with 48 hours of treatment more significant. However, LNCaP cells were more responsive to the nano-Se nanoparticles than DU145 cells for 24 hour treatment while DU145 cells are more responsive for 48 hour treatment. For LNCaP cells, cell viability decreased to 76 to 63% (p<0.05) after 24 hrs of 1-100 μM nano-Se nanoparticle treatment, to 53 to 36% (p<0.05) after 48 hrs of treatment. For DU145 cells, cell viability decreased to 95-80% (p<0.05) after 24 hour treatment, to 37-16% after 48 hours of treatment (p<0.05).

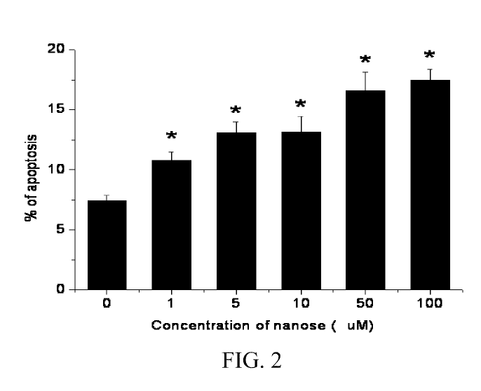

In reference to FIG. 2, nano-Se nanoparticles induced apoptosis was assessed by flow cytometry. Cells (1×10⁶ cells/well) were treated with 1-100 μM nano-Se nanoparticles for 48 hours. Treated cells were then collected and analyzed after staining with annexin V-FITC and propidium iodide (from Sigma).

Apoptotic and necrotic cells were quantified by flow cytometry and quantitative FACS analysis using the annexin V-conjugated Alexa Fluor 488 Apoptosis Detection Kit (from Sigma, USA). The binding of FITC-labeled annexin V to phosphatidylserine that only surfaces on cell membrane during the early phase of apoptosis, was measured for indication of apoptosis. Cells were also stained with propidium iodide (PI), apoptotic cells which generally have intact cell membranes do not stain with PI while necrosis cells which have broken cell membranes do stain with PI. The samples were then subjected to flow cytometry to measure the percentage of apoptotic (FITC-stained cells) and necrotic cells (PI-stained cells). A minimum of 10,000 cells were counted. Flow cytometry assay was performed by using a Coulter Epics XL-MCL (Beckman Coulter, USA).

The percentage of cells that were only positive for annexin V stain is used as a measure of apoptosis. FIG. 2 shows that LNCaP cells exhibited a steady increase of apoptosis when exposing to increasing concentrations of nano-Se nanoparticles for 48 hrs, ranging from about 10.8% of the cells at 10 μM of nano-Se nanoparticle treatment, to 17.5% at 100 μM of nano-Se nanoparticle treatment.

It has been shown that for Se-methylselenocysteine, the β-lyase-mediated production of a monomethylated selenium metabolite from Se-methylselenocysteine is a key step in its cancer chemoprevention. Methylseleninic acid (MSA), a compound that represents a simplified version of Se-methylselenocysteine without the amino acid moiety, thereby obviating the need for β-lyase action has been shown to be more potent than Se-methylselenocysteine in inhibiting cell accumulation and inducing apoptosis in vitro, and has similar effect in vivo. See IP, C., et al, “In vitro and in vivo studies of methylseleninic acid: Evidence that a monomethylated selenium metabolite is critical for cancer chemoprevention,” Cancer research, 2000, vol. 60, No. 11, pp. 2882-2886.

The effect of nano-Se nanoparticles on cell viability is therefore compared with that of MSA, the most potent organic form selenium.

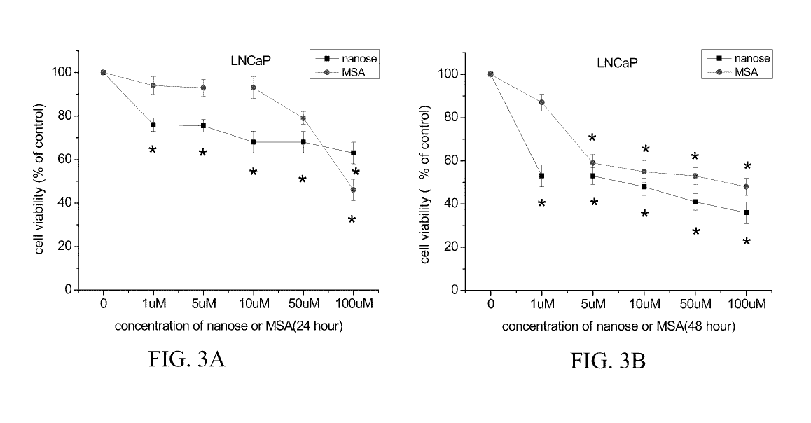

In reference to FIGS. 3A and 3B, 4A and 4B, 5A and 5B, 6A and 6B, the effects of nano-Se nanoparticles on cell viability are compared to that of MSA in androgen dependent prostate cancer line LNCaP cells, androgen independent prostate cancer line DU145 cells, human breast cancer cell line MCF-7 cells, human lung carcinoma cell line A549 cells.

Cells were treated with 0-100 μM nano-Se nanoparticles or MSA for 24 and 48 hours. Cells were then collected and cell viabilities were assayed. The data shown are mean±standard of three samples for each treatment. Differences between groups were analyzed using Student's test with significance assumed at p<0.05.

Both nano-Se nanoparticles and MSA decreased cell viability of LNCaP, DU145, MCF-7 and A549 cells in dose and time-dependent manner. However, for androgen dependent prostate cancer cell line LNCaP cells, nano-Se nanoparticles are more potent than MSA for 48 hour treatment (FIGS. 3A and 3B) while less effective for 24 hour treatment at lower concentration.

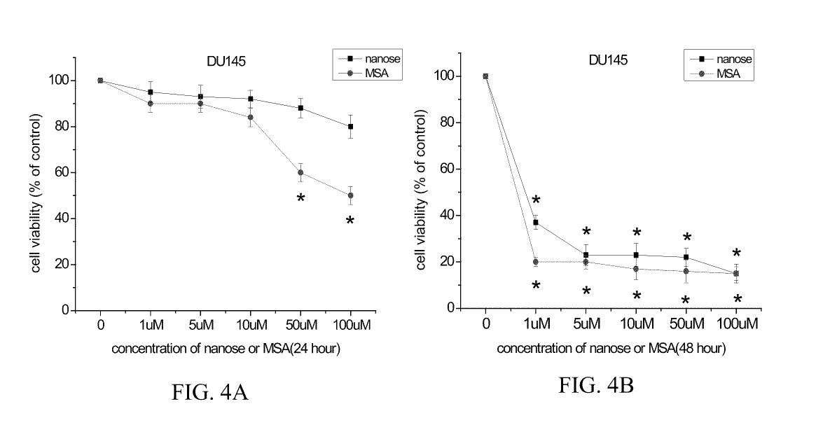

For DU145 cells, the effect of nano-Se nanoparticles and MSA are comparable for 48 hour treatment, but effect of MSA is more significant for 24 hour treatment at all measured concentrations (FIGS. 4A and 4B).

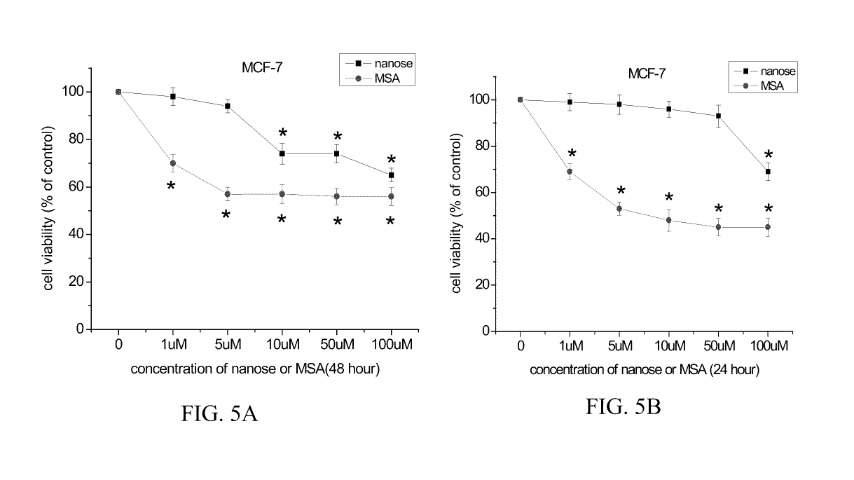

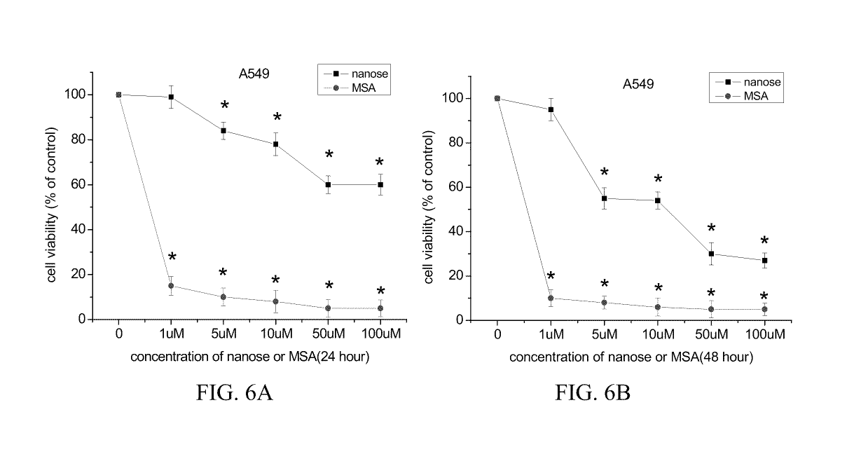

Cell viabilities are decreased in human breast cancer cell line MCF-7 cells and in human lung carcinoma cell line A549 cells with both forms of selenium treatments. But human lung carcinoma cell line A549 cells are significantly more sensitive to the treatments of either agents. However, for both cell types, MSA treatment is more potent in decreasing cell viability (FIGS. 5A, 5B, 6A and 6B).

### Example 2

The underline mechanism of the effect of nano-se nanopartcle is further shown to be androgen and androgen receptor dependent at transcriptional level by using luciferase reporter system, and the measurement of mRNA and protein level of androgen receptor itself.

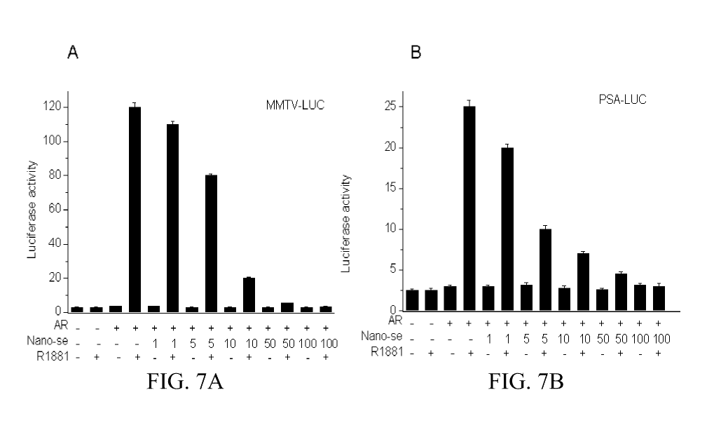

In reference to FIGS. 7A and 7B, HEK293 cells were transiently transfected with mouse mammary tumor virus-luciferase (MMTV-luc) expression vector or pGL3-PSA-luc plasmids (5.85 kb of prostate specific antigen promoter placed upstream of luciferase gene) with or without the co-transfection of the full length human wild type androgen receptor cDNA expression plasmid pAR0. pRL-TK-Luc is used as internal control. Both MMTV-luc and pGL3-PSA-luc plasmids contain Androgen Receptor binding site which recruits androgen receptor and its co-activator to recruit RNA Pol II to the promoter region.

The transfected cells were treated with different concentrations of Nano-Se in the presence or absence of 10 nM R1881 (a potent synthetic androgen) for 24 hours. Cells were then lysed and assayed for luciferase activity using Dual-Luciferase® Reporter Assay System (Promega, Madison, Wis., USA), the experiment was performed in triplicate and repeated at least four times.

As shown in FIGS. 7A and 7B, R1881 induced androgen receptor dependent promoter activity for both MMTV and PSA promoter constructs. This induction was abolished by pretreatment of cells for 1 hour with Nano-Se in a concentration dependent manner.

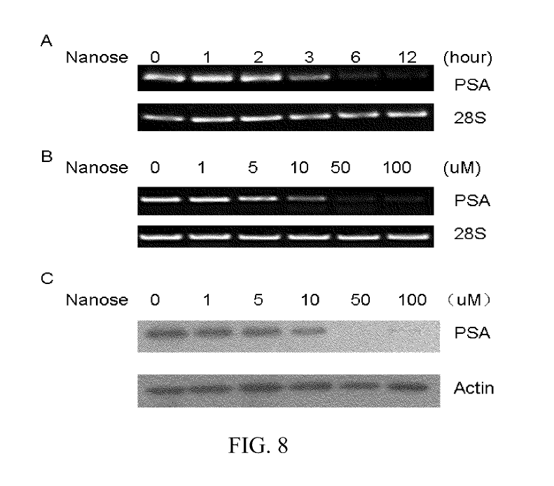

In reference to FIG. 8, the effect of nano-Se nanoparticles was then directly measured on PSA (prostate specific antigen) mRNA level and protein level in LNCaP Cells.

The modulation of PSA mRNA by nano-Se nanoparticles was assessed quantitatively by RT-PCR. And the expression of PSA was measured by Western Blot. Androgen dependent prostate cancer cell line LNCaP Cells were treated with 100 μM nano-Se nanoparticles for various lengths of time. A significant decrease in PSA mRNA was detected as early as 3 hours after exposure to nano-Se nanoparticles as shown in the A panel. As shown in B panel, the depression of PSA mRNA was dependent on the concentration of nano-Se in the range between 1 and 100 μM. In panel C, decrease in PSA protein was evident even with treatment of 1 or 5 μM nano-Se nanoparticles. At 50 or 100 μM Nano-Se, the level of PSA protein became hardly detectable.

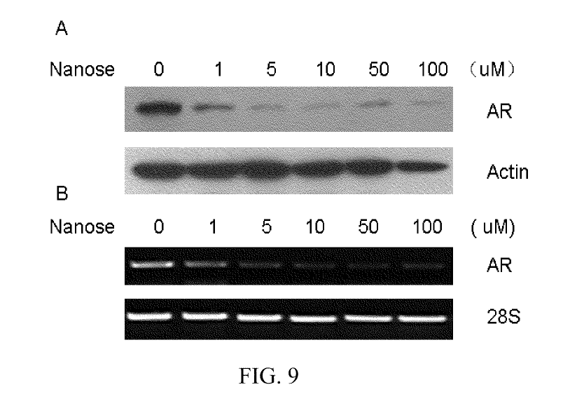

In reference to FIG. 9, the effect of nano-Se nanoparticles was then directly further measured on AR mRNA and protein expression in LNCaP cells.

LNCaP cells were treated for 24 hours with various concentrations of nano-Se nanoparticles. As shown in panel A, in untreated cells, AR protein expression was detectable in unstimulated cells, but was dramatically decreased by 1 μM nano-Se nanoparticle treatment for 24 hours, and the decrease was in a dose dependent manner. In panel B, AR mRNA levels were measured using RT-PCR. LNCaP cells that were not treated with nano-Se expressed high level of AR mRNA, exposure to nano-Se nanoparticles inhibited AR mRNA production in a dose-dependent manner.

As will be recognized by those skilled in the art, the innovative concepts described in the present application can be modified and varied over a tremendous range of applications, for example, selenium nano-particles prepared with agents other than crude peptones. And accordingly the scope of patented subject matter is not limited by any of the specific exemplary teachings given. It is intended to embrace all such alternatives, modifications and variations that fall within the spirit and broad scope of the appended claims.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

What is claimed is:

1. A method for controlling cell growth, comprising the actions of:
exposing the cell to sufficient amount of selenium form from elemental selenium nanoparticles prepared by using biological agents other than pure bovine albumin protein.

2. The method of claim 1, wherein said cell is androgen dependent prostate cancer cells.

3. The method of claim 1, wherein said cell is androgen receptor dependent.

4. The method of claim 1, further comprising the action of manipulating the androgen receptor related regulation mechanism.

5. A chemopreventive method for cancer prohibition, comprising the actions of:
administering patients with sufficient amount of pharmacological composition containing elemental selenium nanoparticles.

6. The method of claim 5, wherein said patients are prostate cancer patients at androgen dependent stage.

7. The method of claim 5, wherein said elemental selenium nanoparticles are prepared by using peptone solution.

8. A chemotherapeutic method for cancer treatment, comprising the actions of:
administering cancer patients with sufficient amount of pharmacological composition containing elemental selenium nanoparticles prepared using biological agents other than pure bovine albumin protein.

9. The method of claim 8, wherein said patients are prostate cancer patients.

10. The method of claim 8, wherein said selenium nano-particles are prepared using crude peptone solutions.
